(19) United States
(12) Patent Application Publication
(10) Pub. No.: US 2025/0186698 A1
(43) Pub. Date: Jun. 12, 2025

(54) REMOVABLY ATTACHABLE ELECTRONIC DOSE MEASURING DEVICE FOR AN INJECTION PEN SYSTEM

(71) Applicant: BIOCORP PRODUCTION S.A., Issoire (FR)

(72) Inventor: Alain MARCOZ, Issoire (FR)

(21) Appl. No.: 18/844,982

(22) PCT Filed: Mar. 9, 2022
(86) PCT No.: PCT/IB2022/000120

§ 371 Date: Sep. 9, 2024

(51) Int. Cl. A61M 5/20; A61M 5/31

(52) U.S. Cl. CPC A61M 5/20; A61M 5/31; A61M 2005/2006; A61M 2205/3327

(57) ABSTRACT

Removably attachable dose measuring device for attachment to a proximal end of an injector pen, comprising: a distal body having a bore and a central longitudinal axis, and adapted for co-axially mounting, and rotationally engaging with, a proximal dose selector wheel of the injector, and comprising at least one magnetic field producing element near a proximal end of the distal body; a proximal dose measuring body, comprising electronic dose measuring circuitry, and mounted within the bore of the distal body, the former being selectively movable axially along, and free to rotate around, the longitudinal axis; wherein the proximal body comprises a temporary positioning means having a magnetizable element, configured to temporarily maintain a fixed spatial, axial and rotational relationship between the distal body and the proximal body along and around the longitudinal axis during attachment and removal of the dose measuring device to, and from, the injector.

The present invention relates to a removably attachable electronic dose measuring device for an injection pen system. As used herein, the terms “pen injection system” and “injection pen system” are used interchangeably to designate a generally handheld pen-shaped injection system, such systems being readily well known per se and commercially available for use in the treatment of many various medical indications. These systems are also often generally designed for self-injection of a drug by the user in need of treatment for the given medical indication. This is for example the case with insulin, supplied in various forms for use in the treatment of diabetes, for example the pen injection systems commercialized under the brand names FlexPen®, as commercialized by Novo Nordisk, Kwikpen®, as commercialized Eli Lilly, or Lantus Solostar®, as commercialized by Sanofi, being but three of the most well known. Other drugs are also used with this category of medical devices, and are required, for example, to address potentially life-threatening situations, enabling immediate emergency injection of a required drug, such as anaphylactic shock treatments, anti-coagulants, opioid receptor agonists and antagonists, and the like, to the extent that it has become a common occurrence for patients suffering from, or susceptible to, such ailments to carry these devices around with them.

With regard to injection pen systems in particular, for example, one of the challenges has been to provide easy to use, reliable and fairly failsafe monitoring and measuring systems that can be adapted to the various different variants of such commercially available pen injection systems, of which there are many. The injection pen system, to which the electronic dose measuring device is adapted and configured for removable attachment, is generally equipped with a proximally located dose setting wheel and an injection activator. The dose setting wheel rotates about a central longitudinal axis of the pen injection system to allow a user to set the dose of medicament for injection. During the dose setting, or dose “dialling” step, the dose setting wheel is generally rotatable in both a clockwise, and a counter-clockwise direction, these directions corresponding generally to an increase in the selected dose, and a decrease in the selected dose, to be administered, respectively, or vice-versa, depending on the manufacturer. The injection activator is often represented by a push-button, usually located proximally of the dose setting wheel, and in the majority of injection pens at the proximal extremity of the injection pen system. After a dose has been set, or “dialled”, as the term is commonly known in the art, when a user of the injection system then presses the injection activator in a distal direction, a piston is driven which is connected to a plunger in order to expel drug from a chamber within the injection pen body out through a needle that the user has inserted into an appropriate injection site, for example, the skin, fatty tissue, or muscle, depending on the type of drug to be administered. The dose setting wheel is sometimes, but not necessarily, also coupled to the injection drive mechanism so that it can, depending on the manufacturer and model of injection pen, also rotate as injection of the drug proceeds. The functioning of such injection systems is well known per se in the art. The monitoring module as envisaged according to the present invention is intended for mounting onto a pen injection system in which the dose setting wheel can be configured to either rotate during the ejection/injection phase of operation, or, on the contrary, not rotate during the ejection/injection phase of operation of the pen injection system. For example, the Kwikpen® injection pen mentioned above does not have a dose setting wheel that rotates during injection, whereas the dose setting wheel of the Lantus Solostar® and FlexPen® injection pens do rotate during injection.

Injection monitoring and dose measuring is known per se when associated with injection pen systems, and enables users of such pen injection systems, and health care professionals involved in the treatment and follow-up of such patients, to monitor more closely their own injection regimes, and in many cases, the doses actually administered, in an attempt to lead to better healthcare outcomes. These developments have been accompanied by the increased associated use of software and portable communications devices such as tablets or smartphones, which have been programmed to receive information from, and interact with, the monitoring and measuring systems in order to provide information to the user or healthcare professional on-the-fly, or at regular intervals via appropriate communications units included in the monitoring systems. The dose measuring device as envisaged in the present specification is adapted and configured to be removably attached to the proximal end of such an injection pen system. The expressions “removably attached”, “removably attachable”, “removably mounted” or “removably mountable” as might be used in the present specification are to be understood as referring to the possibility of attaching, or mounting, and subsequently removing, or dismounting, or unattaching, the dose measuring device, for example, in the case of transferring the dose measuring device to another pen injection system, or for example, if the dose measuring device is damaged during use and requires replacement. Such attachment and subsequent removability can be achieved by providing coupling means on the dose measuring device which engage in a releasable manner with the proximal end of the pen injection system, for example via frictional or elastic engagement, or via other releasable fastening means, such as clips, straps, screw threads and corresponding tightening rings, and the like, which engage with either the dose setting wheel, or the injection activator, and/or even the body of the injection pen system.

The applicant has previously filed a number of patent applications relating to removably attachable electronic dose measuring devices for such injection pen systems, which have been published, for example, WO2020/217076, WO2020/217094, and WO2021/260404A1. Accordingly, the removably attachable electronic dose measuring devices described in these patent applications, can be generally described as being configured and adapted to be removably attached to a proximal end of an injector pen having an injection activation button included at the proximal end of the injector pen to activate injection. More particularly, the electronic dose measuring devices generally comprise the following structure:
a distal cylindrical body having a longitudinal bore and a central longitudinal axis, the distal cylindrical body being adapted and configured for co-axially mounting, and rotationally engaging with, a proximal dose selector wheel located adjacent to, and distally of, the injection activation button of the pen injection system, the distal cylindrical body further comprising at least one magnetic field producing element located at, or adjacent, a proximal end of the distal cylindrical body; and
a proximal electronic dose measuring body, comprising an electronic dose measuring circuitry, the dose measuring body being configured to be mounted within the bore of the distal cylindrical body, and wherein the electronic dose measuring body is selectively movable axially along the central longitudinal axis, and free to rotate about the central longitudinal axis.

Such a structure therefore combines an outer distally located body, which engages with, and rotates with a dose setting wheel of a pen injection system during dose setting, and/or optionally during dose ejection, depending on the specific functioning of the injection pen, and an inner, and proximal body containing an electronic dose measuring circuitry, which inner body is generally free to rotate about central the longitudinal axis, but which is selectively movable axially along said central longitudinal axis within the bore of the distal cylindrical body. The electronic circuitry of the dose measuring body is configured to register changes in magnetic field as the magnetic field producing element is rotated about the central longitudinal axis during dose setting and/or dose ejection, depending on the functioning of the injection pen, but also uses the selective axial movement along the central longitudinal axis to register a change in magnetic moment along the central longitudinal axis to also aid in the determination of other operational conditions, such as the start of an ejection of medicament from the pen injector, the end of an ejection from the pen injector, the duration of an ejection of substance from the injection pen, and the like. In these circumstances, selective axial movement refers to the operation of the injection pen, for example, by indirectly pressing on the activator push button via a corresponding button element provided at a proximal end of the proximal electronic dose measuring body, in order to effect ejection of a substance contained within a cartridge or chamber included in the pen injection system, for example, containing a suitable pharmaceutical or other injectable substance. Similarly, once activation of the push button of the pen injector ceases, for example, due to a user releasing digital pressure on the proximal push button of the electronic dose measuring body, the latter is generally selectively moved in a proximal direction, either through recoil in the pen injection system, or by a return spring provided in the electronic dose measuring body, thereby allowing for the detection or registration by the electronic circuitry of the return to a reset position of the dose measuring device.

In all of the above, the notions of proximal and distal refer to relative positions with regard to pen injection systems in general, and the corresponding removably attachable electronic dose measuring device, wherein, proximal relates to a point or position or direction that is generally oriented in the direction towards the holder of the pen injection system, and distal relates to a point or position or direction that is generally oriented in the direction away from the holder of the pen injection system, for example towards a target site for injection, whether that be another part of the user's body, or a different person's, or animal's, body.

The electronic circuitry contained within the proximal electronic dose measuring body can be quite sensitive to unexpected movement, even when attached to the injection pen system. However, the structure of the electronic dose measuring device has been designed and configured to manage at least some of those unexpected movement circumstances when attached to the pen injector.

It has been determined through actual use of the devices as described above, however, that users of the electronic dose measuring devices are not always as coordinated as could be hoped for when mounting and dismounting the measuring devices to, and from, the proximal end of the injection pen system. In particular, it has been found in practice that users can accidentally cause the electronic dose measuring devices to register events in the electronic circuitry that might be considered as an end of an injection, or as the beginning of an injection, or some other event, such as a failed or incomplete injection, due to relative movement of either the distal cylindrical body, or the proximal electronic dose measuring body.

Accordingly, one aspect of the present invention is to provide a removably attachable electronic dose measuring device as described above, wherein the selectively movable proximal electronic dose measuring body comprises a temporary positioning having a magnetizable element, which temporary positioning means is configured to temporarily maintain a fixed spatial, axial and rotational relationship between the cylindrical body and the electronic dose measuring body along and around the central longitudinal axis during attachment and removal of the electronic dose measuring device to, and from, the proximal end of the injector pen. The temporary positioning means is thus configured to enable the distal cylindrical body and the proximal electronic dose measuring body to be maintained in a fixed spatial and rotational relationship, one with respect to the other while the electronic dose measuring device is being manipulated by a user to attach it, or remove it, from the injection pen.

According to another aspect, the magnetizable element is magnetized by the at least one magnetic field producing element, during attachment to, and before removal from, the proximal end of the injector pen. As used herein, the expression “magnetized by” refers to the fact that the magnetizable element becomes magnetized through magnetic induction of the temporary positioning means, which is caused by the proximity of the magnetic field producing element located at, or adjacent, the proximal end of the distal cylindrical body, with the temporary positioning means on mounting and dismounting of the dose measuring device.

Similarly, and according to yet another aspect, the proximal electronic dose measuring body is configured to assume a first, collapsed position in which the magnetizable element is magnetized, and a second, deployed position in which the magnetizable element is no longer magnetized.

According to a further aspect, in the first, collapsed position, the electronic dose measuring body is positioned in abutting distal surface contact with a proximal surface of the distal cylindrical body, and in the second, deployed position, the electronic dose measuring body has been moved axially along the central longitudinal axis in a proximal direction, and is disengaged from distal surface contact of the electronic dose measuring body with a proximal surface of the distal cylindrical body. It will be understood from the above that the collapsed position as envisaged in the present specification is one in which the distal cylindrical body and the proximal electronic dose measuring body are to all intents and purposes held together in surface-to-surface contact at mutually opposing surfaces of each of the respective bodies, by the attractive force generated between the magnetic field producing element on the one hand, and the induced magnetic field of the temporary positioning means on the other hand. The deployed position, on the contrary, is a position in which said mutually opposing surfaces of the respective distal and proximal bodies are no longer in surface to surface contact, and indeed, are axially spaced apart one from the other, with the proximal electronic dose measuring body being located proximally from the distal cylindrical body, and, more particularly, in a position in which the dose measuring body is free to rotate about the central longitudinal axis.

Magnetic field producing elements are known per se, for example, classical magnets, electromagnets, and mixed material magnets. Such magnets are typically made from magnetizable materials, having magnetic or paramagnetic properties, whether naturally or when an electric or other energizing flow traverses or affects said material to produce or induce a magnetic field in said material. Suitable materials can be appropriately selected from:
ferrite magnets, especially sintered ferrite magnets, for example, comprising a crystalline compound of iron, oxygen and strontium;
composite materials consisting of a thermoplastic matrix and isotropic neodymium-iron-boron powder;
composite materials made up of a thermoplastic matrix and strontium-based hard ferrite powder, whereby the resulting magnets can contain isotropic, i.e. non-oriented, or anisotropic, i.e. oriented ferrite particles;
composite materials made of a thermo-hardening plastic matrix and isotropic neodymium-iron-boron powder;
magnetic elastomers produced with, for example, heavily charged strontium ferrite powders mixed with synthetic rubber or PVC, and subsequently either extruded into the desired shape or calendered into fine sheets;
flexible calendered composites, generally having the appearance of a brown sheet, and more or less flexible depending on its thickness and its composition. These composites are never elastic like rubber, and tend to have a Shore Hardness in the range of about 40 to about 70 Shore D ANSI. Such composites are generally formed from a synthetic elastomer charged with strontium ferrite grains.

The resulting magnets can be anisotropic or isotropic, the sheet varieties generally having a magnetic particle alignment due to calendering;
laminated composites, generally comprising a flexible composite as above, co-laminated with a soft iron-pole plate;
neodymium-iron-boron magnets;
steels made of aluminium-nickel-cobalt alloy and magnetized;
alloys of samarium and cobalt.

Of the above list of magnetic field producing elements suitable for use in the present invention, those selected from the group consisting of neodymium-iron-boron permanent magnets, magnetic elastomers, composite materials made up of a thermoplastic matrix and strontium-based hard ferrite powder, and composite materials made of a thermo-hardening plastic matrix and isotropic neodymium-iron-boron powder, are preferred. Such magnets are known for their ability to be dimensioned at relatively small sizes whilst maintaining relatively high magnetic field strength.

Similarly, and in line with the choice of magnetic field producing element, the magnetizable element of the proximal electronic dose measuring body can be appropriately chosen from those identified above, the difference being that at least initially, the magnetizable element has not been magnetized, or is only magnetized when in sufficiently close proximity to the magnetic field producing element that an induced magnetic field is created in the magnetizable element. As such the magnetic dipole created in the magnetizable element of the proximal electronic dose measuring body can be permanent, semi-permanent, or temporary, and can be suitably configured to be at a maximum when the magnetic field producing element and the magnetizable element are at their closest spatially relative to the central longitudinal axis, and at a minimum once a certain pre-configured distance of separation exists between the proximal electronic dose measuring body and the distal cylindrical body has been reached. This can be configured by suitably choosing the corresponding dimensions, size, and produced magnetic field of the magnetic field producing element.

Whilst the magnetic field producing element can be of any suitable general shape, for example disk-shaped, including circular, ellipsoid, or any other suitable polygonal shape, it preferably has only a single dipole, with a single pair of diametrically opposing north and south magnetic poles. Although the magnetic field producing means can also optionally be substantially disk-shaped, such a disk-shape can also preferably include magnets which have an orifice substantially in the centre of the disk to form a ring or annular shaped magnet. Such a ring or annular shaped magnet can therefore usefully be seated on a peripheral annular and proximal facing surface of the distal cylindrical body at the proximal end thereof. Advantageously, and for the purposes of the presently envisaged configurations, the dipole magnets are rod-shaped or cylindrical dipole magnets, one positioned in opposite polar facing orientation with regard to the other, for example N-S aligning with S-N, whereby the magnets are positioned to lay flat along their own longitudinal axes, across a horizontal plane that bisects, and is orthogonal to, the central longitudinal axis, each magnet being located on an opposing side of said central longitudinal axis, for example, at 180° of rotation around said central longitudinal axis, one with respect to the other.

According therefore to yet another aspect, the magnetizable element of the proximal electronic dose measuring body is located around said central longitudinal axis, on a peripheral surface of the electronic dose measuring body.

According to yet another aspect, the magnetizable element of the proximal electronic dose measuring body is an annular ring. The annular ring can be suitably located around the central longitudinal axis, on a peripheral surface of the electronic dose measuring body, for example, within a peripheral annular groove provided on a peripheral wall of the proximal electronic dose measuring body.

According to another aspect, the magnetizable element is located at least partially on a distal facing peripheral surface of the electronic dose measuring body. For the purposes of understanding, this is deemed to imply that the magnetizable element extends at least partially across a distal facing surface of the dose measuring body. Generally, such a distal facing surface will be located at, or adjacent, a distal end of the dose measuring body, or located on a contour or profile of the dose measuring body that has a distal facing surface. In such a configuration, the distal facing surface on which the magnetizable element is located is positioned in opposition to a mutually and correspondingly positioned proximal facing surface of the proximal end of the distal cylindrical body, for example, in substantial longitudinal axial alignment with the magnetic field producing element or elements.

According therefore to one aspect, the magnetizable element comprises a distal facing surface area of magnetizable material. This distal facing surface area of magnetizable material can appropriately comprise, for example, a magnetizable particulate embedded in a suitable polymer or resin matrix. Alternatively, the distal facing surface area of magnetizable material can be a particulate layer of magnetizable material that has been deposited on, or attached to, via appropriate adhesive or other technically equivalent means, the distal facing surface area.

According to yet another aspect, and most advantageously, the magnetizable element comprising a distal facing surface area of magnetizable material is an insert. The insert can be chosen and configured in shape and dimensions to be physically inserted into the distal facing surface of the dose measuring body, for example, by providing a suitably shaped and dimensioned housing or recess within the distal facing surface area, and can be retained therein by any suitable means, for example by counter-molding, embedding, etc, in a known manner.

According to yet another aspect, the insert comprises a distally oriented face which extends across the distal facing surface area, and which distally oriented face forms a distal end of the insert. Advantageously, and according to yet another aspect, the distal end of the insert is located flush with the distal facing surface area such that the distally oriented face of the distal end does not project distally outwards beyond the distal facing surface area. In this way, the insert and distally facing surface area provide a homogeneous contact surface for mutually opposing contact with the proximal facing surface of the cylindrical body, and more particularly, with the proximal facing surface of the magnetic field producing element.

According to another aspect, the magnetizable insert extends from a first, distal end, to a second, proximal end, the proximal end of the insert being embedded within the electronic dose measuring body. It is to be understood from this that the magnetizable insert extends into, or is enclosed by, or embedded in, a material which forms part of the dose measuring body, or alternatively, and advantageously, can be provided by overmolding such a material onto the measuring body to trap the insert therein.

According to yet another aspect, the magnetizable insert is selected from the group of inserts having a “S”, “Z”, “C” or “U” shape. Alternatively, and advantageously, the magnetizable insert can have a generally annular shape, such as a ring, with a corresponding “S”, “Z”, “C”, or “U”-shaped cross-section or profile. The shape of the insert can be suitably configured and dimensioned according to the degree of stability and reinforcement that it is desired to impart to the magnetizable element within the dose measuring body.

According to yet another aspect, the magnetizable element is positioned in, or on, the electronic dose measuring body at a distal position spaced apart from the electronic circuitry. It is to be understood from this that the magnetizable element is located within the dose measuring body at a distance from the electronic circuitry such that any magnetic field induced in the magnetizable element will not interfere with, or otherwise disturb, the readings operated by the electronic circuitry. Configuring the distance from said circuitry is one way of achieving this, and can be combined with an appropriate choice of strength for the magnetic field producing element.

These and other objects of the invention will become apparent and described in more detail in the following description relating to the figures and an example monitoring module.

### BRIEF DESCRIPTION OF THE FIGURES

The invention will now be described in more detail with regard to the accompanying figures, provided for the purpose of illustration and exemplification, in which:

FIG. 1 is a schematic perspective representation of a first embodiment of an electronic dose measuring device in a deployed position;

FIG. 2 is a schematic cross-sectional representation of another embodiment of an electronic dose measuring device of the invention mounted on the proximal end of a handheld pen injection system in a deployed position;

FIG. 3 is a schematic perspective representation of a distal facing detail of the electronic dose measuring device of FIG. 2;

FIG. 4 is a schematic, exploded perspective representation the detail of FIG. 3;

FIG. 5 is a schematic, perspective representation of a detail of FIG. 4;

FIG. 6 is a schematic, perspective, open cross-sectional representation, or cut-away of the detail of FIG. 4;

FIG. 7 is a schematic, cross-sectional representation of the electronic dose measuring device in the collapsed position;

FIG. 8 is a schematic, cross-sectional representation of the electronic dose measuring device in the deployed position;

FIG. 9 is a schematic, cross-sectional representation of the electronic dose measuring device in the deployed position, after mounting on an injection pen.

### DETAILED DESCRIPTION OF AN EXAMPLE

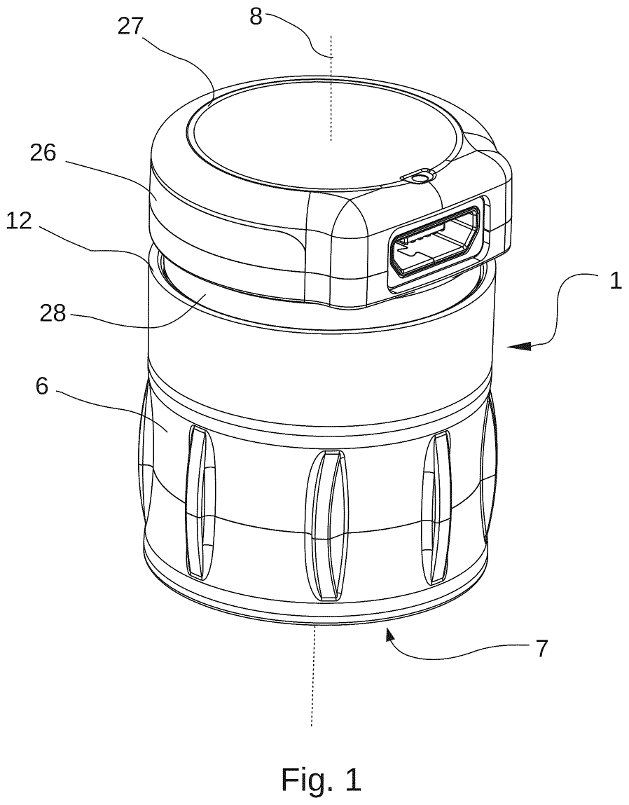

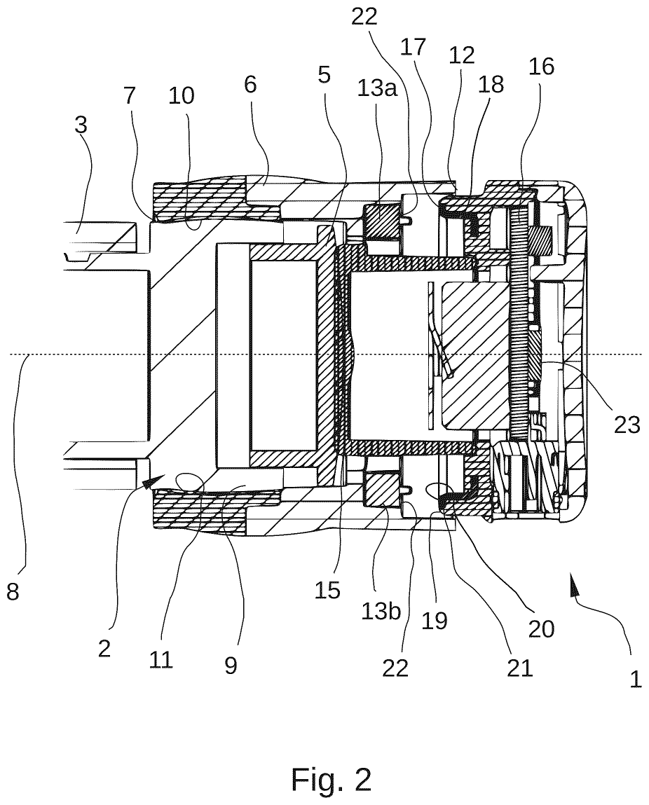

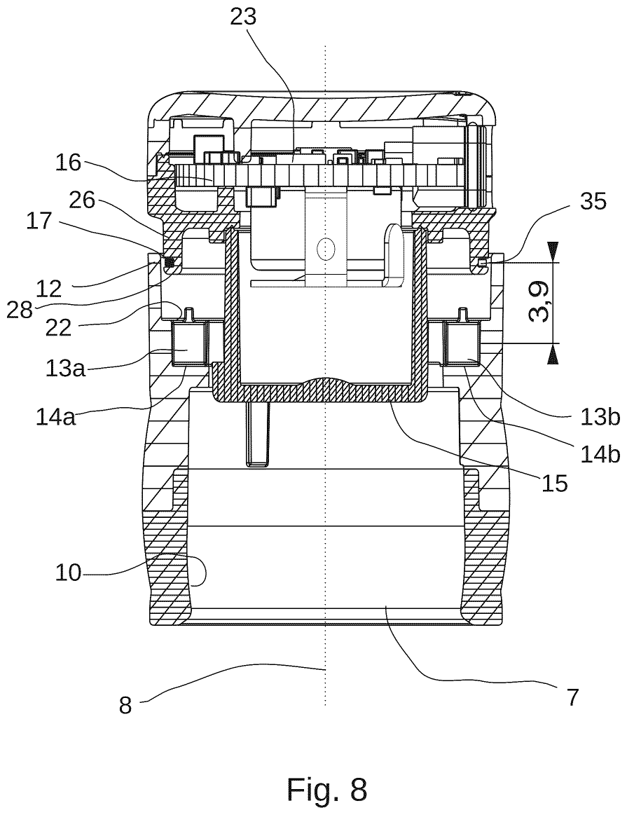

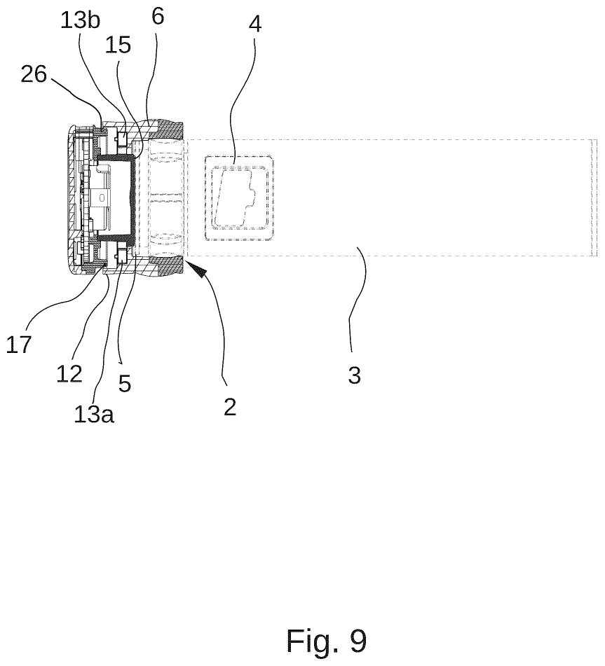

Turning now to FIGS. 1 and 2, a schematic representation of an electronic dose measuring device (1) according to the invention is illustrated in a perspective view in FIG. 1, with the dose measuring device in a deployed position, and a cross-sectional view in FIG. 2, with the dose measuring device in the same deployed position. The electronic dose measuring device (1) in FIGS. 2 and 9 is shown mounted on, or attached to, the proximal end (2) of an injector pen (3) which also shows a dose display window (4), cf. for example FIG. 9. The injector pen (3) has an injection activation button (5, cf. FIGS. 2 & 9) included at the proximal end (2) of the injector pen (3). The electronic dose measuring device (1) comprises a distal cylindrical body (6) having a central longitudinal bore (7) and a central longitudinal axis (8). The distal cylindrical body (6) is adapted and configured co-axially mount onto, and rotationally engage with, a proximal dose selector wheel (9) located adjacent to, and distally of, the injection activation button (5). Rotational engagement of the distal cylindrical body (6) with the dose selector wheel is commonly achieved through a snap-fit or friction fit of an inner surface (10) of the distal cylindrical body onto an outer surface (11) of the dose selector wheel (9), such that any rotational movement imparted to the distal cylindrical body imparts a corresponding rotational movement to the dose selector wheel about the central longitudinal axis to select a dose for injection in the pen injector. At, or near the proximal end (12) of the distal cylindrical body (6), a pair of N-S dipole magnets (13a, 13b) is provided to form the magnetic field producing element. Alternatively, the pair of dipole magnets can be advantageously replaced by an annular magnet having a single dipole or multiple pairs of N-S poles, in which the annular magnet would have a central bore aligned with the central longitudinal axis (8) and the central bore (7) of the distal cylindrical body (6). In FIG. 2, it can be seen that the magnets are located within the bore (7) of the distal body (6) and seated within a suitably shaped housing or recesses (14a, 14b) that forms an integral part of the distal cylindrical body (6). A proximal electronic dose measuring body (15) comprising an electronic dose measuring circuitry (16) is mounted at least partially within the bore (7) of the distal cylindrical body (6). The electronic dose measuring body (15) is selectively movable axially along the central longitudinal axis (8), and is free to rotate about the central longitudinal axis in the deployed position illustrated in FIGS. 1, 2, 8 and 9.

The selectively movable proximal electronic dose measuring body (15) also comprises a temporary positioning means (17), which is configured to temporarily maintain a fixed spatial, axial and rotational relationship between the cylindrical body and the electronic dose measuring body along and around the central longitudinal axis during attachment and removal of the electronic dose measuring device to, and from, the proximal end of the injector pen. The temporary positioning means (17) is designed to temporarily fix the proximal electronic dose measuring body (15) to the distal cylindrical body (6) whilst the electronic dose measuring device (1) is being mounted onto the proximal end (2) of the pen injection system (3). The temporary positioning means (17) thereby enables the proximal electronic dose measuring body (15) to assume a first, collapsed position in which the magnetizable element is magnetized, and which holds the proximal dose measuring body (15) against the distal cylindrical dose setting body (6), in abutting distal surface contact with a proximal surface of the distal cylindrical body (6), via the attractive force of opposing magnetic poles located respectively in the magnetic field producing element, on the one hand, and the magnetizable element of the temporary positioning means, on the other hand. Furthermore, the temporary positioning means also enables the proximal dose measuring body (15) to assume a second, deployed position in which the magnetizable element is no longer magnetized, and in which the proximal dose measuring body (15) has been moved axially along the central longitudinal axis (8) in a proximal direction and is disengaged from distal surface contact of the electronic dose measuring body (15) with a proximal surface of the distal cylindrical body (6).

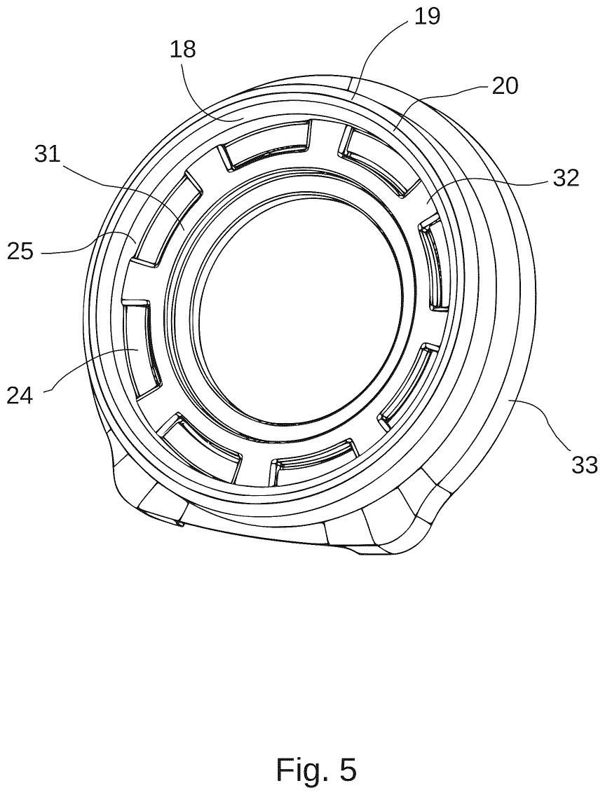

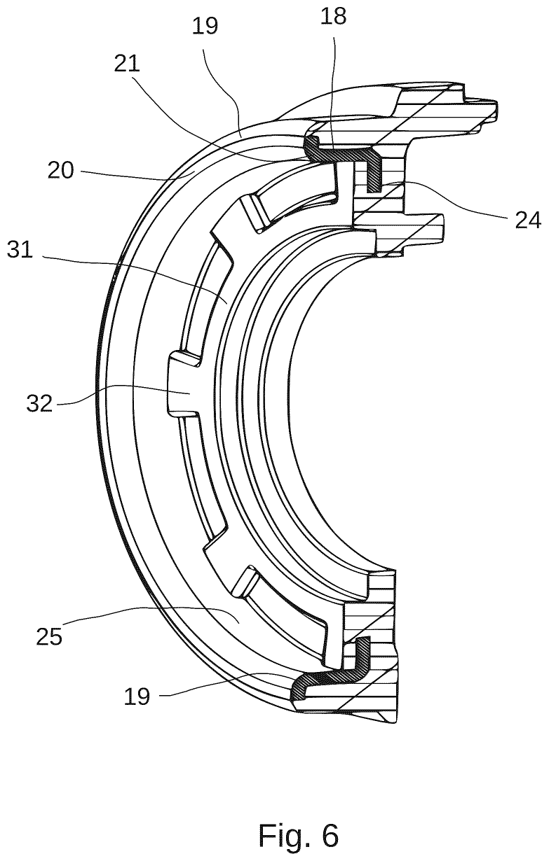

As can be seen from FIGS. 2, 3, 4, 5 and 6, according to one embodiment, the magnetizable element of the temporary positioning means (17) is an insert (18) having a generally “S” or “Z”-shaped profile or cross-section (cf. FIG. 2 and FIG. 6) and is located around the central longitudinal axis (8), and extends at least partially on and over a distal facing peripheral surface (19) of the electronic dose measuring body (15). Alternatively, and in another advantageous embodiment (not shown), the magnetizable element comprises a distal facing surface area (19) of magnetizable material, such as a magnetizable coating or particulate material which can be deposited onto, or integrated into the peripheral distal facing surface of the dose measuring body (15). Such magnetizable elements can usefully also be provided as molded resin matrices containing magnetizable particles. As illustrated in FIGS. 2 and 5, the insert (18) comprises a distally oriented face (20) which extends across the distal facing surface area (19), the distally oriented face (20) forming a distal end (21) of the insert (18). The distal end (21) of the insert is located flush with the distal facing surface area (19) such that the distally oriented face (20) of the distal end (21) does not project distally outwards beyond the distal facing surface area (19), thereby providing an essentially smooth distal contact surface area for contacting a proximal facing contact surface (22) of the distal cylindrical body (6). As will be understood from the figures, the proximal facing contact surface (22) of the distal cylindrical body (6) is comprised of a proximal facing surface of the magnetic field producing element (13a, 13b). Prior to mounting of the electronic dose measuring device (1) to the injection pen, the proximal dose measuring body (15) is pushed towards the distal cylindrical body (6), for example by holding the distal cylinder (6) and proximal measuring body (15) between finger and thumb, and bringing finger and thumb together. Doing so brings the distal facing surface area (19) of the dose measuring body (15), and distal facing surface (20) of the insert (18), into proximity with the magnets (13a, 13b). The magnetic fields produced by the magnets (13a, 13b) induce an opposite, and attractive magnetic field in the insert (18). The attractive force draws the opposing surfaces (19, 20, 22) of the respective proximal measuring body (15) and distal cylindrical body (6) together into temporary surface abutment, and which corresponds to the collapsed position. In this collapsed position, the proximal dose measuring body (15) is no longer free to rotate about the central longitudinal axis (8), nor can it further translate in a direction that would bring it further into the bore (7) of the distal cylindrical body (6). Mounting of the measuring device (1) onto the proximal end of the injection pen can now proceed, safe in the knowledge that the proximal dose measuring body will not rotate around the central longitudinal axis (8) and potentially induce an erroneous reading in the electronic circuitry, which can contain, among others, a magnetometer (23), as known in the art from published PCT applications WO2020/217076, WO2020/217094. The deployed position, in which mutual surface abutment, and correspondingly magnetic attractive force, between the distal cylindrical body (6) and the proximal dose measuring body (15) is no longer engaged, is obtained by exerting a proximally directed force to the dose measuring body (15), thereby overcoming the magnetic attractive force that held the two bodies together. Such a force can be exerted by the user, for example, by simply pulling on the dose measuring body along the central longitudinal axis. Alternatively, an actuatable spring biasing element can be provided to assist in moving the proximal dose measuring body in a proximal direction. In doing so, the proximal dose measuring body (15) translates axially along the central longitudinal axis into the deployed position, in which the dose measuring body (15) is once again free to rotate about the central longitudinal axis.

As illustrated in FIGS. 2 and 6, the magnetizable insert (18) extends from a first, distal end (21), to a second, proximal end (24), the proximal end (24) of the insert being embedded within the electronic dose measuring body (15). The insert (18) can be chosen and configured in shape and dimensions to be physically inserted into the distal facing surface area (19) of the dose measuring body (15), for example, by providing a suitably shaped and dimensioned housing or recess within the distal facing surface area (19), and can be retained therein by any suitable means, for example by counter-molding, embedding, etc. As illustrated more particularly in FIGS. 2 and 6, the magnetizable insert (18) extends from the distal end (21) via a central portion (25) to the proximal end (24), which extends into, or is enclosed by, or embedded in, the material which forms part of the dose measuring body (15). Alternatively, the insert (18) can be provided already pre-integrated into, or forming part of, an insertable module suitably dimensioned and configured to engage with the measuring body (15) in push, snap or press fit engagement and be retained in said measuring body (15).

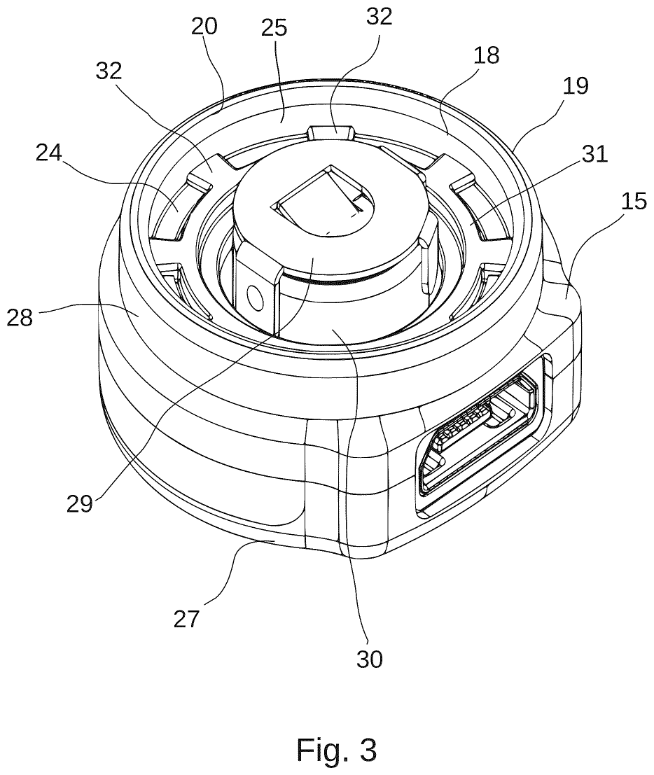

FIGS. 3, 4 and 5 illustrate some of the elements of the dose measuring body (15), whereby FIG. 3 illustrates the dose measuring body as it would appear upside down, with the distal facing surfaces apparent. As can be seen from FIG. 3, the dose measuring body (15) is defined by a peripheral body wall (26) which extends from a proximal end (27) towards a distal end (28). The distal end (28) of the peripheral body wall (26) is configured to present a diameter which allows it to fit, and translate, within the bore (7) of the distal cylindrical body (6). The dose measuring body (15) comprises a number of other elements that are visible in FIG. 3 which relate to the functioning of the electronic circuitry (16), such as a battery holder (29), and corresponding battery (30), to supply electrical power to the electronic circuitry. FIG. 3 also illustrates more clearly the distal surface area (19), the distal facing surface (20) and distal end (21) of the insert (18), as well as the central portion (25) of the insert (18) extending from the distal end (21) towards the proximal end (25), and the flush alignment between the distal surface (20) of the insert (18) and the distally facing surface area (20) of the dose measuring body (15). As can further be seen from FIG. 3, the insert (18) is retained within the dose measuring body by a molded, overmolded or integrated hub (31) and spoke (32) structure which comprises a central hub (31) located around the battery holder (29) and from which a series of spokes extend radially outwardly to cover and/or overlap with a part of the proximal end (24) of the insert, thereby retaining and stabilizing the insert within the dose measuring body (15). In this manner, the magnetizable element, as illustrated here, the insert (18), is positioned in, or on, the electronic dose measuring body (15) at a distal position spaced apart from the electronic circuitry (16), and that this spaced apart distance is sufficient to ensure that the insert, will not interfere with the readings that are carried out by the electronic circuitry (16).

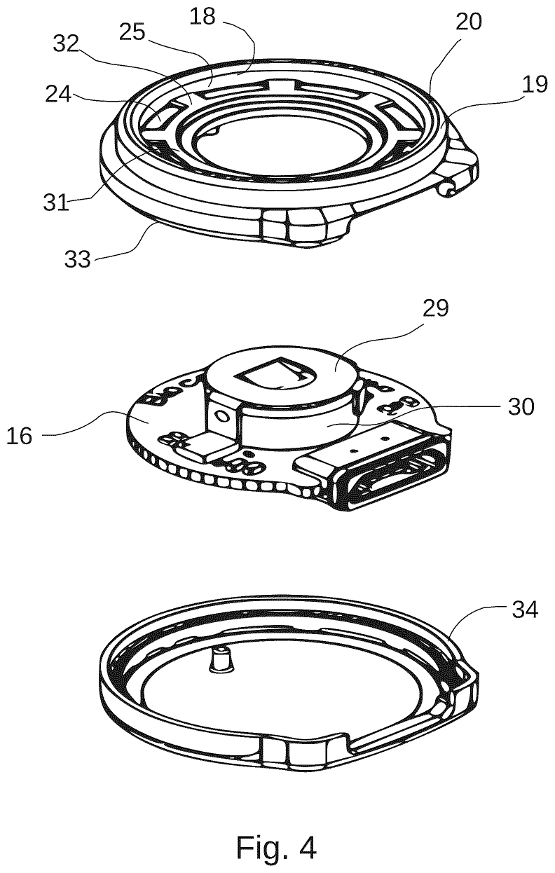

FIG. 4 illustrates an exploded perspective representation of the dose measuring body (15), in which it can be seen that the electronic circuitry (16) is sandwiched between a distal body component (33) and a proximal body component (34), which can be snap-fit, or push-fit, or ultrasonically welded together.

Similarly, FIG. 5 illustrates a magnified representation of the distal body component (33) showing the insert (18) with its distal facing surface (20) and distal end (21) and the respective distal facing surface area of the dose measuring body (19), along with the central portion (25) of the insert and proximal end (24) in relation to the hub (31) and spokes (32) enclosing and retaining the insert (18) within the dose measuring body (15).

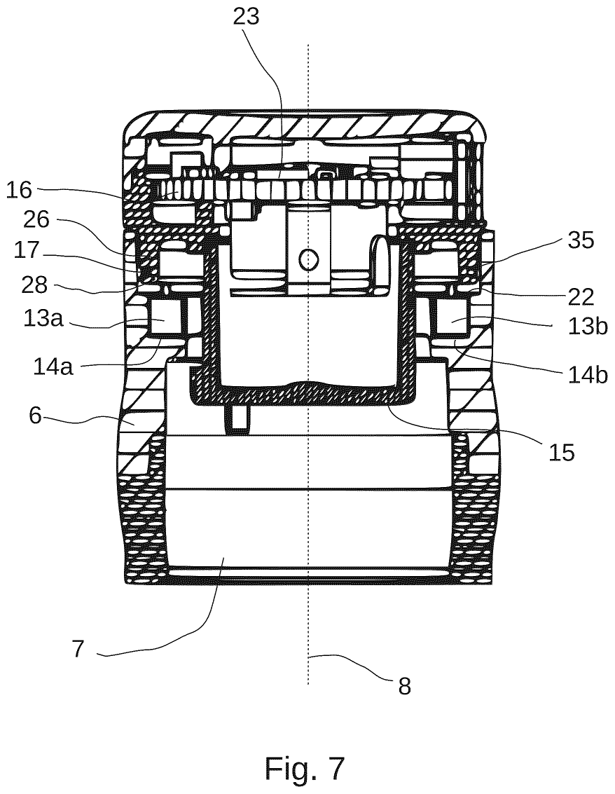

FIGS. 7, 8 and 9 bear the same reference numerals for like parts of the electronic dose measuring device (1), and illustrate an alternative embodiment in which the temporary positioning means (17) is embodied by an annular ring (17). The annular ring (17), which may be a whole ring, or a split-ring, for example, having either no end overlap, with the possibility of a gap between each opposing facing end, or else some overlap of the ends of the ring. The ring (17) is seated within a peripheral annular groove (35) provided on an outward facing surface of the peripheral body wall adjacent the distal end of the dose measuring body (15) is defined by a peripheral body wall (26) which extends from a proximal end (27) towards the distal end (28) of the dose measuring body (15). The annular ring is comprised of a magnetizable material in the same, or similar way, as the insert (18), and functions in an identical manner to the insert (18) with regard to the relative movements of the distal, cylindrical, dose setting body (6) and the proximal, electronic, dose measuring body (15), and the movement into the collapsed position for mounting and dismounting of the device from the injection pen, and the movement into the deployed position once the dose measuring device has been mounted on the injection pen. The main difference in this embodiment is that the magnetizable surface of the ring (18) does not come into direct contact with the proximal facing surface (22) of the magnets (13a, 13b), even though a distal facing surface area (19) does come into contact with said proximal facing surface (22) in the collapsed position.

1. Removably attachable electronic dose measuring device, configured and adapted to be removably attached to a proximal end of an injector pen having an injection activation button included at the proximal end of the injector pen to activate injection, the removably attachable electronic dose measuring device comprising:
a distal cylindrical body having a longitudinal bore and a central longitudinal axis, the distal cylindrical body being adapted and configured for co-axially mounting, and rotationally engaging with, a proximal dose selector wheel located adjacent to, and distally of, the injection activation button, the distal cylindrical body comprising at least one magnetic field producing element located at, or adjacent, a proximal end of the distal cylindrical body;
a proximal electronic dose measuring body, comprising an electronic dose measuring circuitry, the dose measuring body being configured to be mounted within the bore of the distal cylindrical body, wherein the electronic dose measuring body is selectively movable axially along the central longitudinal axis, and free to rotate about the central longitudinal axis;
wherein the selectively movable proximal electronic dose measuring body comprises a temporary positioning means having a magnetizable element, which temporary positioning means is configured to temporarily maintain a fixed spatial, axial and rotational relationship between the cylindrical body and the electronic dose measuring body along and around the central longitudinal axis during attachment and removal of the electronic dose measuring device to, and from, the proximal end of the injector pen.

2. Removably attachable electronic dose measuring device according to claim 1, wherein the proximal electronic dose measuring body is configured to assume a first, collapsed position in which the magnetizable element is magnetized, and a second, deployed position in which the magnetizable element is no longer magnetized.

3. Removably attachable electronic dose measuring device according to claim 3, wherein in the first, collapsed position, the electronic dose measuring body is positioned in abutting distal surface contact with a proximal surface of the distal cylindrical body, and in the second, deployed position, the electronic dose measuring body has been moved axially along the central longitudinal axis in a proximal direction, and is disengaged from distal surface contact of the electronic dose measuring body with a proximal surface of the distal cylindrical body.

4. Removably attachable electronic dose measuring device according to claim 1, wherein the magnetizable element is magnetized by the at least one magnetic field producing element, during attachment to, and before removal from, the proximal end of the injector pen.

5. Removably attachable electronic dose measuring device according to claim 2, wherein the magnetizable element of the proximal electronic dose measuring body is located around said central longitudinal axis, on a peripheral surface of the electronic dose measuring body.

6. Removably attachable electronic dose measuring device according to claim 2, wherein the magnetizable element of the proximal electronic dose measuring body is an annular ring.

7. Removably attachable electronic dose measuring device according to claim 6, wherein the annular ring is located within a peripheral annular groove provided on a peripheral wall of the proximal electronic dose measuring body.

8. Removably attachable electronic dose measuring device according to claim 2, wherein the magnetizable element is located at least partially located on a distal facing peripheral surface of the electronic dose measuring body.

9. Removably attachable electronic dose measuring device according to claim 2, wherein the magnetizable element comprises a distal facing surface area of magnetizable material.

10. Removably attachable electronic dose measuring device according to claim 2, wherein the magnetizable element comprising a distal facing surface area of magnetizable material is an insert.

11. Removably attachable electronic dose measuring device according to claim 10, wherein the insert comprises a distally oriented face which extends across the distal facing surface area, the distally oriented face forming a distal end of the insert.

12. Removably attachable electronic dose measuring device according to claim 10, wherein the distal end of the insert is located flush with the distal facing surface area such that the distally oriented face of the distal end does not project distally outwards beyond the distal facing surface area.

13. Removably attachable electronic dose measuring device according to claim 10, wherein the magnetizable insert extends from a first, distal end, to a second, proximal end, the proximal end of the insert being embedded within the electronic dose measuring body.

14. Removably attachable electronic dose measuring device according to claim 10, wherein the magnetizable insert is selected from the group of inserts having a “S”, “Z”, “C” or “U” shape.

15. Removably attachable electronic dose measuring device according to claim 2, wherein the magnetizable element is positioned in, or on, the electronic dose measuring body at a distal position spaced apart from the electronic circuitry.
